(19) United States
(12) Patent Application Publication
(10) Pub. No.: US 2023/0404552 A1
(43) Pub. Date: Dec. 21, 2023

(54) BIOPSY DEVICES AND METHODS

(71) Applicant: Dilon Medical Technologies LTD., Caesarea (IL)

(72) Inventor: Yoav ZUR, Atlit (IL)

(21) Appl. No.: 18/319,005

(22) Filed: May 17, 2023

Related U.S. Application Data
(60) Provisional application No. 63/343,660, filed on May 19, 2022.

(51) Int. Cl. A61B 10/02

(52) U.S. Cl. CPC A61B 10/0275

(57) ABSTRACT

A biopsy system includes a handle for receiving a removable biopsy device which includes an inner needle having a distal tip configured for piercing tissue, a tissue sensing unit for characterizing tissue properties, a recess formed longitudinally in the inner needle proximal to the distal tip for receiving a tissue portion from a region of interest, an outer sheath configured to move relatively to the inner needle, and a connector element for receiving sensing signals from the tissue sensing unit. The handle includes a socket tiltable between a rest position and a tilted position, and having a sliding channel, and a connection board. The connector element includes a coupling matrix while the connection board has a receiving matrix. Fixation pins in one matrix cooperate with holes in a second matrix to facilitate alignment of the connector element with the connection board.

### TECHNOLOGICAL FIELD

The present disclosure relates to surgical medical devices, and in particular to devices for obtaining a tissue sample in vivo.

### BACKGROUND

In core biopsy procedures, the biopsy device typically consists of an inner needle, or a pointed tip stylet, which includes a sampling cavity close to the needle tip. The sample cavity is in the form of a trough, or a shallow receptacle, covered by an outer sheath, cannula or sleeve having a cutting edge. The device is provided with a mechanism enabling relative movement between the inner needle and the sheath to allow acquiring a tissue sample in the sampling cavity.

During a biopsy procedure, the biopsy device is inserted into the body, preferably in a closed state, where the sheath is in a forward position covering the sampling cavity. The biopsy device has also an open state, where the sheath is in a retracted position relative to the inner needle revealing the sampling cavity.

When approaching the biopsy sampling location, the inner needle is fired forwardly inside the body towards the biopsy sampling location to reveal the sampling cavity. The outer sheath, being provided with cutting means, is then moved forwardly so as to cut the tissue that fills the needle trough (i.e. the sampling cavity) thereby enclosing the tissue in the needle trough.

Instead of or in addition to externally applied diagnostic imaging systems, such as X-ray or Ultrasound, aimed at guiding the device inside the body or locating tissue of interest to be sampled, some biopsy devices include sensor(s) aimed at characterizing the tissue in vivo, for example so as to improve the localization of tissue of interest and the overall sampling procedure.

US2018042586, assigned to the assignee of the present invention, discloses a biopsy device which includes an inner needle comprising a tip for piercing tissue, and a cavity for receiving a tissue portion. It also includes an outer sheath moving with respect to the inner needle between a forward position of the sheath in which it totally covers the cavity and a backward position in which the cavity is totally uncovered. The outer sheath includes a cutting edge at its front end configured to cut the tissue portion into the cavity while moving in a forward direction. It also includes a sensing unit of the tissue and a suction system for applying suction force in the cavity to pull the tissue portion into the cavity while the sheath moves in a backward direction.

U.S. Pat. No. 9,488,192, assigned to the assignee of the present invention, discloses a surgical tool for use in a tissue removal procedure from a subject. The surgical tool has proximal and distal regions and at least one sensor for sensing one or more predetermined conditions located at a distal region of the surgical tool. The sensors are connected by a transmission structure to the proximal region of the biopsy device.

WO2009010960, assigned to the assignee of the present invention, discloses a medical device for use in tissue characterization and treatment. The device comprises a tissue characterization probe comprising an elongated carrier for carrying an array of tissue characterization sensors arranged in a spaced-apart relationship at least along an axis of said carrier, such that progression of the probe through a tissue mass provides for locating and determining a dimension of an abnormal tissue specimen inside said tissue mass based on characterization signals from the sensors in the array, thereby enabling consequent treatment of the abnormal tissue specimen by a treatment tool.

### General Description

The needle and sheath of the biopsy device are in direct contact with the tissue of a patient. In order to avoid cleaning and disinfection, it may be advantageous that the biopsy device be made disposable. Thus, it is an object of the current disclosure to present a removable biopsy device, and in particular to have a sensing unit which connects easily and removably to a handle of a biopsy system.

According to one aspect, a biopsy system which includes a handle for receiving a removable biopsy device and the removable biopsy device are provided. The biopsy system may further include a processing console (also referred to as control unit) and a display screen. The biopsy device includes an inner needle having a distal tip configured for piercing tissue, and a tissue sensing unit configured for characterizing tissue properties. Part of the tissue sensing unit may be positioned on the inner needle. The biopsy device may also include a recess formed longitudinally in the inner needle proximal to the tip for receiving a tissue portion from a region of interest, an outer sheath configured for changing of axial position relative to the inner needle, and a connector element for receiving sensing signals from the tissue sensing unit.

The outer sheath may change axial position relative to the inner needle between a forward position in which the sheath covers (optionally totally) the recess and a backward position of the sheath in which the recess and the part of the tissue sensing unit are uncovered (optionally totally). The outer sheath may include a cutting edge at its distal end for cutting and enclosing the tissue portion into the recess when moving relatively to the needle from the backward position to the forward position.

The handle may comprise a registration assembly which includes a socket tiltable between a rest position and a tilted position. The socket may have a sliding channel configured to receive the connector element when the socket is in the tilted position. In other words, when the socket is in the titled position, an opening of the sliding channel may be exposed for allowing insertion of the connector element. The socket may further include a connection board configured for electrically coupling the connector element when the socket is in the rest position and the connector element is received in the sliding channel.

In some embodiments, the tissue sensing unit includes sensors positioned at a distal portion of the inner needle, and a flat transmission strip connected to the sensors and to the connector element. Preferably, the sensors are impedance controlled tissue characterization sensors. Preferably, a sensor is positioned at the tip of the inner needle. Most preferably, a groove is made at the tip for accommodating therein a sensor, such that an upper surface of the sensor does not protrude above outer surface of the tip.

Preferably, an array of space-apart sensors is positioned in the recess. Preferably, part of the flat transmission strip is fixedly attached to a surface of the inner needle.

In some embodiments, the biopsy device (i.e. the needle and tissue sensing unit) is disposable.

In some embodiments, the connector element includes a coupling matrix of spaced apart sensor couplers. Preferably, the registration assembly includes a connection board which has a receiving matrix for attachment to the coupling matrix, such as to receive and transmit the signals from and to sensors. Preferably, the registration assembly includes an alignment mechanism for spatial alignment between the coupling matrix and the receiving matrix. Most preferably, the alignment mechanism is configured to slide the connector element and position the receiving matrix with the coupling matrix to enable impedance controlled attachment between them. Most preferably, the alignment mechanism is configured to align between the coupling matrix and the receiving matrix by pressing one against another.

Preferably, the connection board is fixed to a housing element of the handle. Preferably, the connector element comprises a plurality of fixation pins or holes associated structurally to the coupling matrix, and the connection board includes matching holes or pins associated structurally to the receiving matrix, thereby facilitating three-dimensional alignment of the connector element with the connection board. Alternatively, the connector element has conical pins configured to enter respective conical hollow receivers at the connection board.

In some embodiments, the handle has a two-part housing, a first part which includes a movement mechanism and an installation mechanism for installing a proximal portion of the biopsy device in the handle, and a second part which hosts the registration assembly.

In some embodiments, the tiltable socket is associated to a housing of the handle by a pivot allowing its tilting.

In some embodiments, the handle is shaped elongated and has a transverse rectangular cross-section along a portion of its length, wherein the cross-section has a perimeter of 14-25 cm. A total weight of the handle and the biopsy device is less than 400 gr.

In some embodiments, the biopsy system communicates with a control unit to transmit input data indicative of signals from the tissue sensing unit. Consequently, the control unit analyzes the signals, and generates output data to inform a user about a tissue portion condition and thus to enable acquisition of a tissue sample.

In some embodiments, the biopsy system includes a sensor sensing a location of at least one sensor of the tissue sensing unit. Also, the biopsy system includes a sensor sensing orientation and/or moving status of the biopsy device.

In some embodiments, the tip has an attached sensor and is protruding from the sheath in the forward position. Alternatively, a distal end of the tip is aligned axially with the sheath edge in the forward position.

In some embodiments, the sheath is free of impedance sensors.

According to one aspect, a method for operating a biopsy system which includes a handle and a biopsy device is provided. The biopsy device has an inner needle with a tip for piercing tissue, a recess formed longitudinally in the inner needle, an outer cutting sheath having a cutting edge, and a tissue sensing unit which has a transmission strip connecting sensors attached to a distal portion of the inner needle to a connection element. The method includes positioning a proximal end of a biopsy device into the handle, attaching a coupler matrix of the connector element to a receiving matrix of a connection board inside the handle, infiltrating the biopsy device into a tissue, reading sensed data regarding a tissue portion proximate to a tip and/or recess of the inner needle, and pushing the outer cutting sheath such that its cutting edge cutting a tissue portion which in turn entering the recess.

In some embodiments, the method includes opening a cover of the handle, sliding the connection element inside a tiltable socket before attaching the matrices, and closing the cover of the handle.

In some embodiments, the method further includes tilting the tiltable socket from a rest position to a tilted position configured to enable sliding the connection element therein, and tilting the tiltable socket from the tilted position to the rest position thereby attaching the matrices to each other.

In some embodiments, the method further includes sensing a location of sensors, and sensing orientation and/or moving status of the biopsy device.

According to one aspect, a mating structure for connecting a removeable connector to a connection board is provided. The removeable connector communicates signals with a plurality of sensors, and the multi-use connector communicates the signals to a control unit.

The mating structure includes a coupler matrix which includes space-apart couplers on a flat side of the removeable connector, a receiving matrix on a face of the connection board for mating with the coupler matrix by fixation pins matching respective holes, and a socket tiltable relative to the receiving matrix between a rest position and a tilted position, configured to slidably receiving the removeable connector therein.

In some embodiments, the coupler matrix includes fixation pins, and the receiving matrix includes respective holes. Some fixation pins couple electrically with respective holes. Alternatively, the receiving matrix includes fixation pins, and the coupler matrix includes respective holes.

In some embodiments, a biopsy system as described above has a connector element receiving sensing signals from the tissue sensing unit and deliver the signals to a control unit using the mating structure.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A schematically illustrates a biopsy system comprising a handle and a biopsy device connected to a control unit.

FIG. 1B presents the handle of FIG. 1A in an open position.

FIG. 2 presents a disposable biopsy device.

FIG. 3A depicts an inner needle of the biopsy device.

FIG. 3B illustrates a tissue sensing unit of the biopsy device.

FIG. 3C illustrates the distal portion of the biopsy device having an attached sensor array in a recess and a sensor in a tip of a needle. An outer cutting sheath envelops the back part of the needle.

FIG. 3D shows the outer cutting sheath covering the recess in a forward position.

FIG. 4 illustrates a distal portion having a sensor only on a tip of the needle according to an embodiment of the current disclosure.

FIG. 5A-B illustrate respectively an inner needle without and with a sensor array on a recess of the needle according to an embodiment of the current invention.

FIG. 6A shows a side view of a socket of a registration assembly attached axially to a cover of the handle while its body is fixed to an element of the handle cover.

FIG. 6B shows a side view of the socket in a tilted position, ready to slidably receive a connector array of the biopsy device.

FIG. 6C shows a front view of the registration assembly and the connection element with the socket in a tilted position.

FIG. 6D shows a connection board fixed to an element of the cover of the handle.

FIG. 6E is a perspective view of the registration assembly with a connection element inside the tilted socket above the connection board.

FIG. 6F is an enlargement of a portion of FIG. 6E showing a coupling matrix of the connection element above a receiving matrix the connection board.

FIG. 6G shows a conical alignment pin and a respective conical receiver.

FIG. 7 is a block diagram of a mating structure connecting a removable connection element with a registration assembly.

FIG. 8 is a flowchart of a method for the biopsy system.

FIG. 9A shows a user sliding the connector element inside the socket.

FIG. 9B shows a user holding the handle using a single hand and infiltrating the biopsy device into a patient breast.

FIG. 9C shows a user pushing a biopsy sample out of the needle recess into a dish.

### DETAILED DESCRIPTION OF EMBODIMENTS

It is to be understood that the present disclosure is not limited to the disclosed example embodiments. It should also be understood that not every feature of the methods and systems handling the system is necessary to implement the present disclosure as claimed in any particular claim of the appended claims. Various elements and features of devices are described to fully enable the present disclosure. It should also be understood that throughout this disclosure, where a method is shown or described, the steps of the method may be performed in any order or simultaneously, unless it is clear from the context that one step depends on another being performed first. In addition, a step may be repeated several times.

Before explaining several embodiments of the present disclosure in detail, it is to be understood that the present disclosure is not limited in its application to the details of construction and the arrangement of the components set forth in the following description or illustrated in the drawings. The present disclosure is capable of other embodiments or of being practiced or carried out in various ways. Also, it is to be understood that the phraseology and terminology employed herein is for the purpose of description and should not be regarded as limiting.

Unless otherwise defined, all technical and scientific terms used herein have the same meaning as commonly understood by one of ordinary skill in the art to which this present disclosure belongs. The systems, methods, and examples provided herein are illustrative only and not intended to be limiting.

In the description and claims of the present application, each of the verbs “comprise”, “include” and “have”, and conjugates thereof, are used to indicate that the object or objects of the verb are not necessarily a complete listing of members, components, elements or parts of the subject or subjects of the verb.

The terms “inner” and “outer” and their derivatives such as “inward” and “outward” may be defined with reference to a longitudinal axis of the needle, wherein an element which is radially closer to the longitudinal axis than another element is referred to as inner while referred to as outer if it is farther.

The terms “proximal” and “distal” may be used to refer to a relative proximity to the handle of the biopsy system. An element may be referred to as distal if it is further away from the handle than another element which can then be referred to as proximal

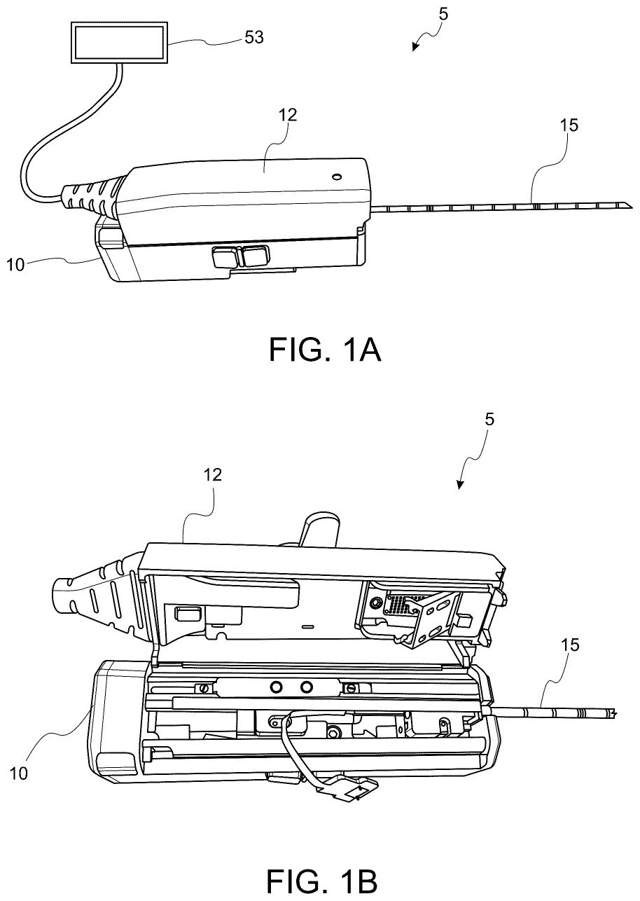

Biopsy System Embodiments (FIGS. 1A-B, 2, 3A-D, 4, 5A-B, 6A-G)

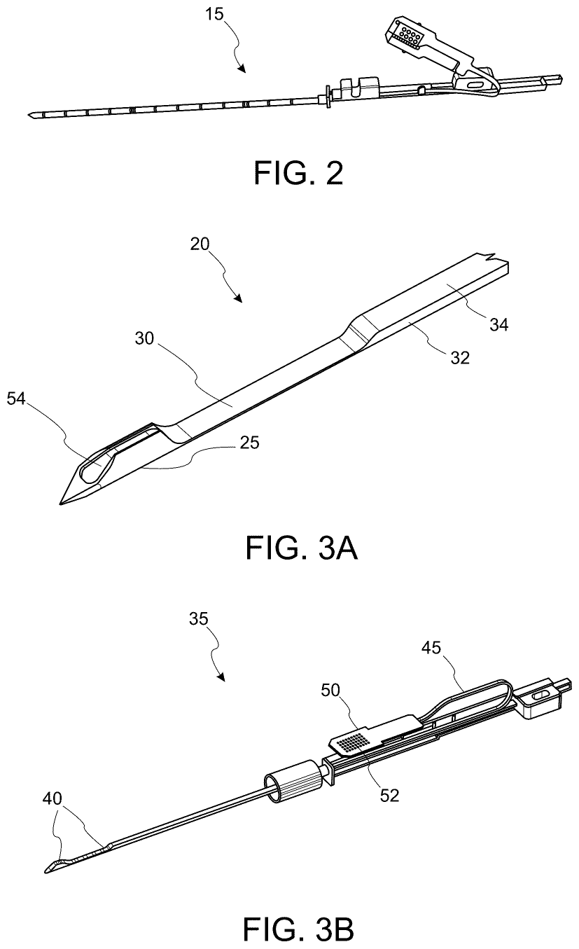

A biopsy system 5 which includes a handle 10 and a removable biopsy device 15 is illustrated schematically in FIGS. 1A and 1B. The handle is shown closed in FIG. 1A and with a cover 12 open in FIG. 1B. The removable biopsy device 15, fully shown in FIG. 2, may also be disposable.

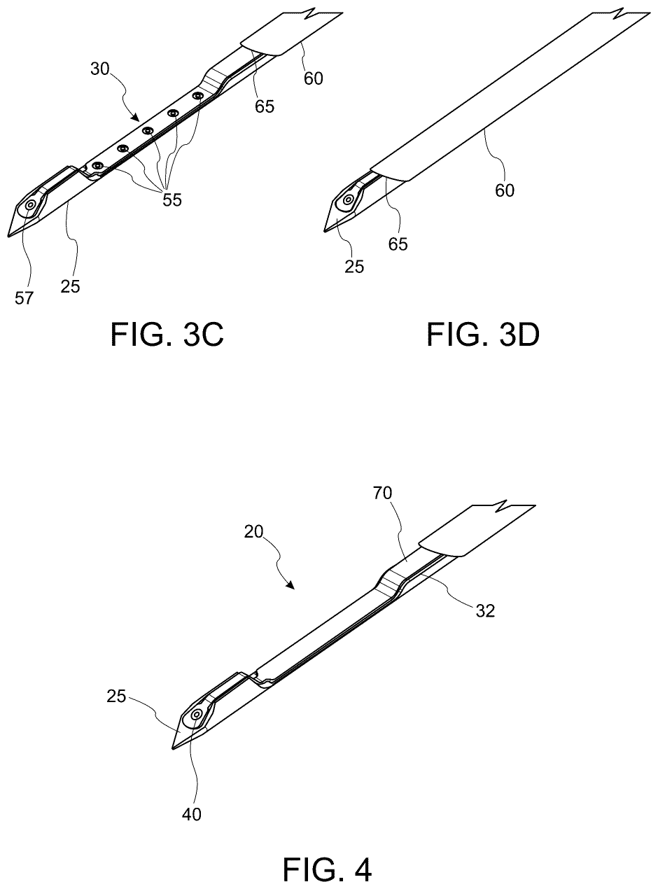

The biopsy device 15 is further shown in FIGS. 3A-3D. FIG. 3A depicts a distal portion of the biopsy device 15. It includes an inner needle 20 having a tip 25 for piercing a tissue and a recess 30 formed longitudinally in the inner needle 20 for receiving a tissue portion from a region of interest. The recess 30 is connected to the proximal part 32 of the inner needle 20, which has a flat top face 34.

FIG. 3B illustrates a tissue sensing unit 35 for characterizing tissue properties. The tissue sensing unit 35 includes a plurality of sensors 40 being positioned at a distal portion of the inner needle as detailed below, and a flat transmission strip 45 connected on one end to the plurality of sensors 40 and on the other side to a connector element 50. A coupler matrix 52 resides on a face of connector element 50, as further detailed below.

The biopsy system 5 may be configured to communicate with a control unit 53, shown in FIG. 1A, so as to transmit input data indicative of signals from the tissue sensing unit 35 to the control unit 53. The control unit 53 may analyze the signals, and generate output data to inform a user about a tissue portion condition. This may prompt the user to proceed to acquisition of a tissue sample. Details of an exemplary sensors for tissue condition appear in international patent application WO2009010960, assigned to the assignee of the present disclosure, and incorporated here for all purposes.

The biopsy needle 20 may comprise a groove 54 at the tip 25, as shown in FIG. 3A. FIG. 3C illustrates the needle 20 with a sensor array 55 positioned fixedly (attached) in the recess 30 and a sensor 57 in the groove 54, such that an upper surface of the sensor 57 does not protrude above outer surface of the tip 25. The sensors 40 and 57 may be impedance controlled tissue characterization sensors, as in WO2009010960. The sensing unit 35 may be disposable as a part of a disposable biopsy device 15.

An outer cutting sheath 60 may envelop the proximal (back) part of the needle as shown in FIG. 3C. The outer sheath 60 may be able to change position relative to the inner needle 20 between a backward position of the sheath 60 in which the recess 30 and a part of the tissue sensing unit 35 are uncovered, as shown in FIG. 3C, and a forward position in which the sheath 60 totally covers the recess 30, as shown in FIG. 3D. The outer sheath 60 may include a cutting edge 65 at its distal end for cutting a tissue portion. The outer sheath 60 may enclose the tissue portion into the recess 30 when moving relative to the needle 20 from the backward position to the forward position.

As shown in FIG. 3D, in the forward position of the outer cutting sheath 60, the tip 25 may be protruding from the sheath 60. Alternatively, the tip 25 may be aligned axially (not shown) with the sheath edge 65 which covers all of it or most of it.

FIG. 4 illustrates a needle 20 which has a sensor 40 only on the tip 25. A part 70 of the flat transmission strip 45 may be fixedly attached to a surface of the inner needle, both on the recess 30 and on a flat face 34 of the part 32 of the needle 20.

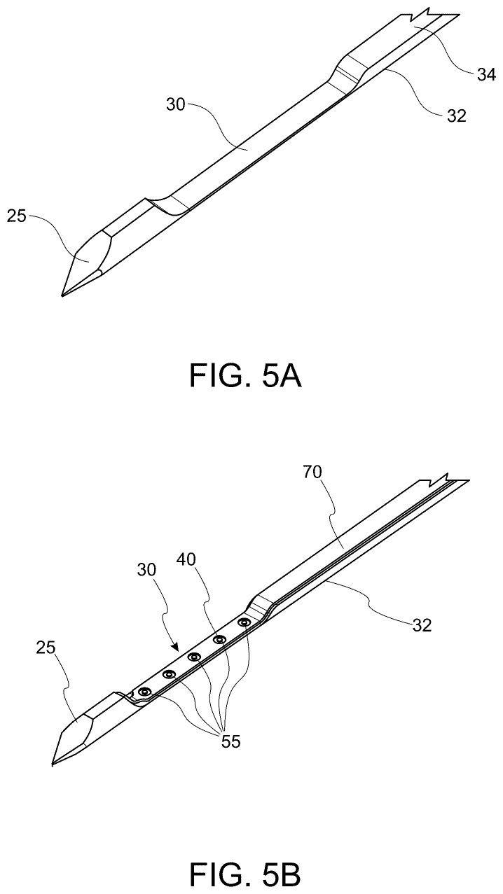

In the example of FIG. 5A, the tip 25 has no groove for receiving a sensor, but an array 55 of space-apart sensors 40 may be positioned in the recess 30 as shown in FIG. 5B.

The handle 10 may have a two-part housing. A first part of the housing may include a movement mechanism and an installation mechanism for installing a proximal portion of the biopsy device in the handle. A second part of the housing, associated with the cover 12, may host a registration assembly 75. Exemplary movement mechanism and installation mechanism are described in patent publication US2018042586, assigned to the assignee of the present disclosure, and incorporated here for all purposes.

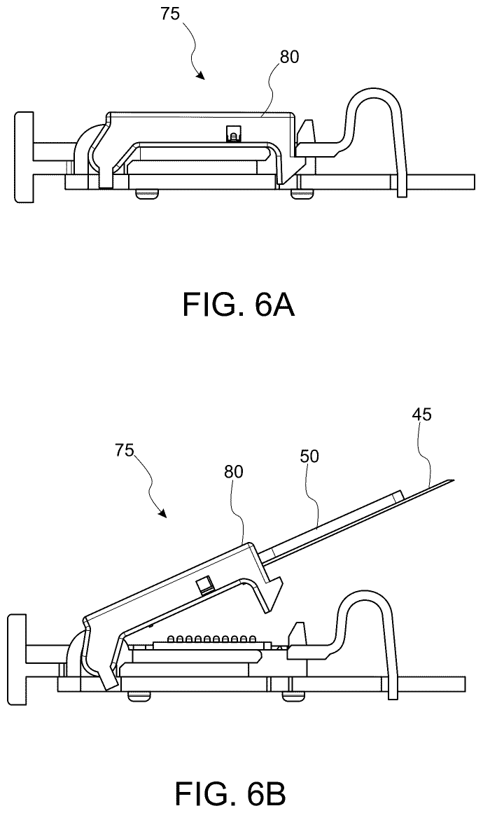

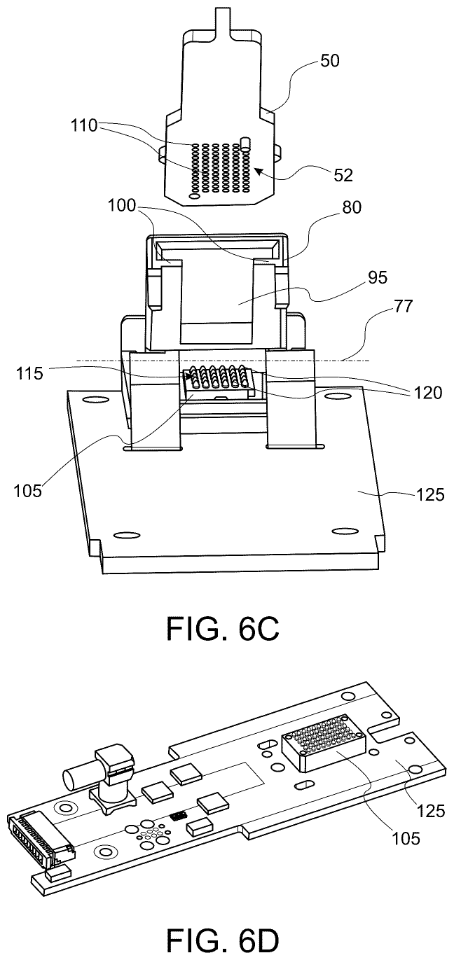

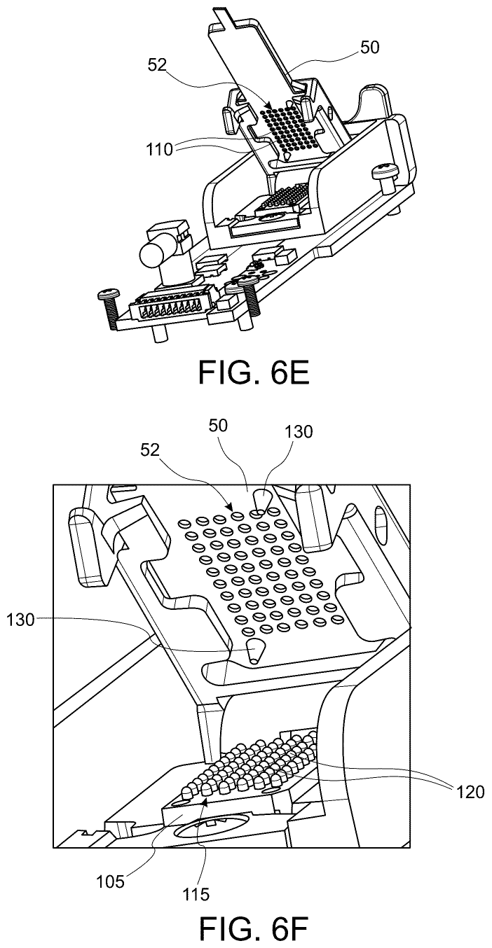

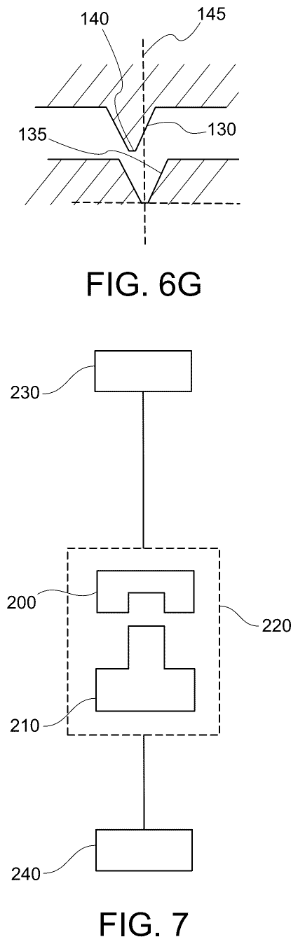

The registration assembly 75 may include a socket 80 tiltable around a pivot 77 between a rest position, as shown in FIG. 6A and a tilted position, as shown in FIGS. 6B, 6C, 6E and 6F. The axis of pivot 77 is illustrated in FIG. 3C.

The socket 80 may include a sliding channel between a flat surface 95 and side walls 100 forming a slot. The registration assembly 75 may also include multi-use connection board 105.

As shown in FIG. 6D, the multi-use connection board 105 may be stationary, and may be fixed to a housing element 125 of the cover 12 of handle 10.

In the example of FIG. 6C and FIG. 6F, the coupler matrix 52 of connector element 50 may include a plurality of columns (e.g. 6 in the example of FIG. 6C and FIG. 6F) and a plurality of rows (e.g. 10 in the example of FIG. 6C and FIG. 6F) of spaced-apart sensor hole couplers 110. Respectively, the multi-use connection board 105 includes a receiving matrix 115 which hosts a number of fixation pin connectors 120 corresponding to the number of sensors (e.g. 60 in the example of FIG. 6C and FIG. 6F). At least part of the hole couplers 110 and fixation pin connectors 120 may be configured to couple electrical signals.

As shown in FIGS. 6C, 6E, and 6F, the registration assembly 75 may include alignment mechanisms to spatially align the coupler matrix 52 and the receiving matrix 115. A first alignment mechanism may be formed by the above sliding channel of the socket 80. A second alignment mechanism may be formed by the pressing of the coupler matrix 52 against the receiving matrix 115 while tilting the socket 80 back to the rest position, such that each fixation pin connector 120 penetrates a corresponding hole coupler 110.

In some embodiments, the coupler matrix 50 may have fixation pin couplers (not shown), similar to the fixation pins 120, while the receiving matrix 105 may have hole connectors (not shown), similar to the hole couplers 110.

A third alignment mechanism is shown in the enlarged view of FIG. 6F and in the schematic drawing of FIG. 6G. The connector element 50 may have two truncated conical pins 130 configured to engage respective two truncated conical hollow receivers 135 at the registration board 105. While tilting the socket 80 back to the rest position, a central point 140 of a flat tip of the truncated conical pin 130 may approach the intersection of a vertical symmetry axis 145 of the hollow receiver 135 with a plane 150 which is perpendicular to axis 145, ensuring three-dimensional alignment of the coupler matrix 52 with the receiving matrix 115 of the connection board 105.

The handle 10 may be shaped as an elongated element and may have a transverse rectangular cross-section along a portion of its length. The cross-section perimeter is in the range of 14-25 cm. A total weight of the handle and the biopsy device is less than 400 gr. The size and weight of the handle 10 facilitate gripping it with only one hand of a user.

The biopsy system 5 may include a sensor sensing a location of a sensor 40. This may enable to detect a location of the biopsy needle during insertion in a body. In some embodiments, the biopsy system includes external imaging system such as X-ray or ultrasound imaging capability.

The biopsy system 5 may include a sensor sensing orientation and/or moving status of the biopsy device 15. In particular, the biopsy device may include an accelerometer configured to detect a movement direction. The biopsy device may also include a sensor (e.g. a light sensor) configured to detect if the socket is in the rest position or in the tilted position. The biopsy device may also include a sensor configured to detect if the handle is open or closed. The biopsy device may also include a sensor to detect the position of the inner needle relative to the outer sheath. In some embodiments, a feedback (e.g. visual, audio or tactile) indicative of an output detection of any of said sensors may be provided to a user of the biopsy device. The feedback may for example be provided on the display screen of the biopsy system.

In contrast to the inner needle 20, the sheath 60 may be free of impedance sensors or any other sensor.

Mating Structure Embodiment (FIGS. 1B, 2, 3B, 6A-G, 7)

Referring now to the block diagram of FIG. 7, a removeable connector element 200 and a connection board 210 may constitute together a mating structure 220 that may be used in mating the removeable connector element 200 or 50 to a connection board 210 or 105. The removeable connector element 200 may communicate signals from a plurality of sources 40 in a first device 230 or 15. The connection board 210 may communicate the signals to a second device 240 or 53.

The mating structure 220 may include a coupler matrix 52 which has space-apart couplers 110 on a flat side of the removeable connection element 200, and a receiving matrix 115 on a face of the connection board 210. The receiving matrix 115 may be configured for mating with the coupler matrix 52 by fixation pins 115 matching respective holes 110. The mating structure 220 may further include a socket 80 tiltable relative to the receiving matrix 115 between a rest position and a tilted position. The socket 80 may be configured to slidably receiving the removeable connection element 200 therein.

The removable connector element 200 may be a part of a disposable unit while the connection board 210 is a multi-use board.

The mating structure 220 may be a part of a biopsy system 5 as explained above with regard to FIGS. 1B, 2, 3B and 6A-G.

A Method for a Biopsy System (FIGS. 1A-B, 2, 3A-D, 4, 5A-B, 6A-G, 8, 9A-C)

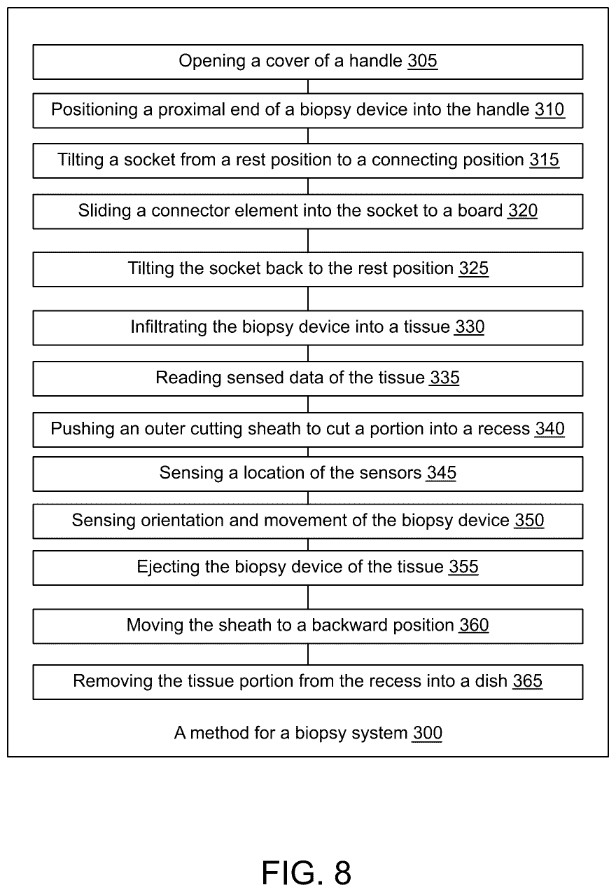

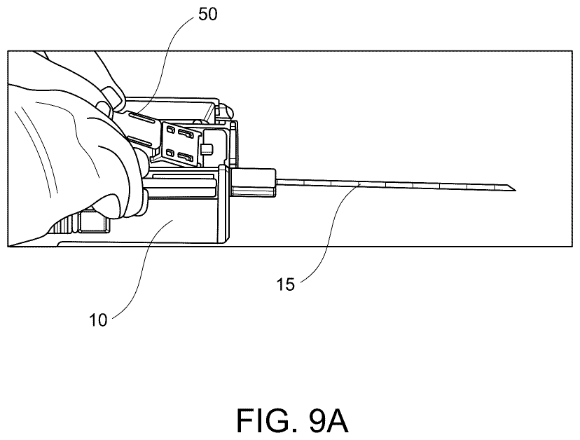

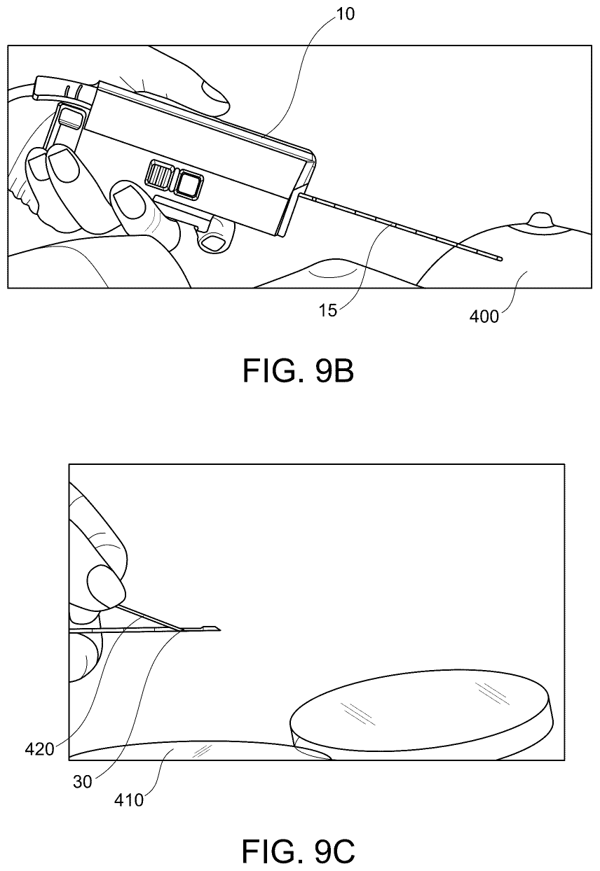

According to one aspect, a method 300 for operating a biopsy system 5 is provided, as presented in a flowchart of FIG. 8. The method 300 may include a step 310 of positioning a proximal end of a biopsy device 15 into the handle 10, a step 320 and a step 325 of attaching a coupler matrix 52 of the connector element 50 to a receiving matrix 115 of a connection board 105 inside the handle 10, and a step 330 of infiltrating the biopsy device 15 into a tissue or breast 400, as shown in FIG. 9B.

The method 500 may further include the step 335 of reading sensed data regarding a tissue portion proximate to a tip 25 and/or recess 30 of an inner needle 20 of the biopsy device 15, and a step 340 of pushing an outer cutting sheath 60 such that its cutting edge cutting a tissue portion (not shown) which in turn enters the recess 30.

The method 300 may also include a step 305 of opening a cover 12 of the handle a step of closing the cover 12 of the handle 10, and a step 320 of sliding the connection element 50 inside a tiltable socket 80 as shown in FIG. 9A.

The method 300 may further include a step 315 of tilting the tiltable socket 80 from a rest position to a tilted position (also referred to as connecting position) to enable the step 320 of sliding the connection element 50 therein, and a step 325 of tilting the tiltable socket back from the tilted position to the rest position thereby attaching the matrices 52 and 115 to each other.

The method 300 may further include a step 345 of sensing a location of sensors 40 using a location sensor.

The method may further include a step 350 of sensing orientation and/or moving status of the biopsy device using an orientation/moving sensor.

The method 300 may also include a step 355 of ejecting the biopsy device 15 of the tissue 400, a step 360 of moving the sheath 60 to a backward position, shown in FIGS. 3C and 4, and a step 365 of removing the tissue portion from the recess 30 into a dish 410 using a sliver 420, as shown in FIG. 9c.

Those skilled in the art will readily appreciate that various modifications and changes can be applied to the embodiments of the present disclosure as hereinbefore described without departing from its scope defined in and by the appended claims.

1. A biopsy system comprising:
(a) a removable biopsy device comprising:
an inner needle comprising a tip configured to pierce tissue and a tissue sensing unit for characterizing tissue properties, at least part of said tissue sensing unit being positioned on said inner needle,
a recess formed longitudinally in said inner needle proximal to said tip and configured for receiving a tissue portion from a region of interest; and
an outer sheath configured to change position relative to said inner needle between a forward position of said sheath in which said sheath covers the recess and a backward position of the sheath in which said recess and said at least part of said tissue sensing unit are uncovered, said outer sheath comprising a cutting edge at its distal end configured to cut and enclose said tissue portion into said recess when moving relatively to the needle from the backward position to the forward position;
a connector element configured for receiving sensing signals from said tissue sensing unit; and
(b) a handle for receiving said removable biopsy device; said handle comprising a registration assembly including:
a socket tiltable between a rest position and a tilted position, and having a sliding channel for receiving said connector element; and
a connection board for electrically coupling said connector element when the connector element is received in the sliding channel and the socket is in the rest position.

2. The biopsy system of claim 1, wherein said tissue sensing unit includes: one or more sensors positioned at a distal portion of said inner needle and a transmission strip connected to said one or more sensors and to said connector element.

3. The biopsy system of claim 2, wherein at least one of said one or more sensors is an impedance controlled tissue characterization sensor.

4. The biopsy system of claim 2 wherein a sensor is positioned at said tip of said inner needle.

5. The biopsy system of claim 4, wherein said inner needle comprises a groove at said tip configured to accommodate therein at least said at least one tissue characterization sensor, such that an upper surface of the sensor does not protrude above outer surface of said tip.

6. The biopsy system of claim 2 wherein an array of space-apart sensors is positioned in said recess.

7. The biopsy system of claim 2 wherein at least part of said flat transmission strip is fixedly attached to a surface of said inner needle.

8. The biopsy system of claim 1, wherein said biopsy device is disposable.

9. The biopsy system of claim 1 wherein said connector element includes a coupling matrix of spaced apart sensor couplers.

10. The biopsy system of claim 9, wherein said registration assembly comprises a connection board which includes a receiving matrix configured to be attached to said coupling matrix to receive and transmit said signals from and to said one or more sensors.

11. The biopsy system of claim 10, wherein said registration assembly comprises an alignment mechanism configured to spatially align between said receiving matrix and said coupling matrix.

12. The biopsy system of claim 11, wherein said alignment mechanism is configured to slide said connector element and position said receiving matrix with said coupling matrix to enable impedance controlled attachment between them.

13. The biopsy system of claim 11, wherein said alignment mechanism is configured to align between said coupling matrix and said receiving matrix by pressing one against another.

14. The biopsy system of claim 10, wherein said connection board is fixed to a housing element of said handle.

15. The biopsy system of claim 10, wherein said connector element comprises a plurality of fixation pins or holes associated structurally to said coupling matrix, and said connection board includes matching holes or pins associated structurally to said receiving matrix to thereby enable the transmission of signal from and to the sensors.

16. The biopsy system of claim 10, wherein said connector element has at least one conical pin configured to enter at least one respective conical hollow receiver at the connection board thereby facilitating three-dimensional alignment of the connector element with the connection board.

17. The biopsy system of claim 1, wherein said handle is configured as a two-part housing comprising a first part comprising a movement mechanism configured for enabling relative movement between the inner needle and the outer sheath and an installation mechanism configured for installing a proximal portion of the biopsy device in the handle, and a second part comprising said registration assembly.

18. The biopsy system of claim 1, wherein said tiltable socket is associated to a housing element of the handle by a pivot allowing tilting of said tiltable socket.

19. The biopsy system of claim 1 wherein the handle is shaped elongated and has a transverse rectangular cross-section along a portion of its length, the cross-section having a perimeter of 14-25 cm.

20. The biopsy system of claim 1 wherein a total weight of said handle and said biopsy device is less than 400 gr.

21. The biopsy system of claim 1, configured to communicate with a control unit to transmit to the control unit input data indicative of signals from said tissue sensing unit, thereby causing the control unit to analyze said signals, and generate output data to inform a user about a tissue portion condition and enable acquisition of a tissue sample.

22. The biopsy system of claim 1 wherein the biopsy system includes a sensor sensing a location of at least one sensor of said tissue sensing unit.

23. The biopsy system of claim 1 wherein the biopsy system includes a sensor sensing orientation and/or moving status of said biopsy device.

24. The biopsy system of claim 1, wherein the tissue sensing unit includes one or more sensors positioned at a distal tip of said inner needle and said distal tip is protruding from said sheath in said forward position.

25. The biopsy system of claim 1, wherein a distal end of said tip is aligned axially with the sheath edge in said forward position.

26. The biopsy system of claim 1, wherein said sheath is free of impedance sensors.

27. A method for operating a biopsy system comprising a handle and a biopsy device having an inner needle comprising a tip configured to pierce tissue, a recess formed longitudinally in said inner needle, an outer cutting sheath having a cutting edge, and a tissue sensing unit having a transmission strip connecting one or more sensors attached to a distal portion of said inner needle to a connection element, the method comprising:
(a) positioning a proximal end of a biopsy device into said handle;
(b) securing a coupler matrix of the connector element to a receiving matrix of a connection board inside said handle;
(c) inserting the biopsy device into a tissue;
(d) reading sensed data regarding a tissue portion proximate to a tip and/or recess of said inner needle; and
(e) pushing said outer cutting sheath such that the cutting edge cuts a tissue portion which in turn enters said recess.

28. The method of claim 27 further comprising steps of:
A. opening a cover of said handle;
B. closing said cover of said handle; and
C. sliding said connection element inside a tiltable socket before securing the coupler matrix to the receiving matrix.

29. The method of claim 28 further comprising a step of tilting said tiltable socket from a rest position to a tilted position configured to enable sliding said connection element therein, and a step of tilting said tiltable socket from the tilted position to the rest position thereby securing the coupler matrix to the receiving matrix.

30. The method of claim 27 further comprising sensing a location of at least one of said one or more sensors.

31. The method of claim 27 further comprising sensing orientation and/or moving status of said biopsy device.

32. A mating structure for connecting a removeable connector element to a connection board, the removeable connector element configured to communicate signals from a plurality of impedance controlled tissue characterization sensors, the multi-use connection board configured to communicate the signals to a control unit, the mating structure comprising:
(a) a coupler matrix including couplers disposed on a flat side of said removeable connector;
(b) a receiving matrix on a face of said connection board configured for mating with the coupler matrix by fixation pins matching respective holes; and
(c) a socket tiltable relative to the receiving matrix between a rest position and a tilted position, configured to slidably receiving the removeable connector therein.

33. The mating structure of claim 32, wherein said coupler matrix includes fixation pins and the receiving matrix includes respective holes, at least one fixation pin is configured for electrical coupling with at least one respective hole.

34. The mating structure of claim 32, wherein the receiving matrix includes fixation pins and the coupler matrix includes respective holes, at least one fixation pin is configured for electrical coupling with at least one respective hole.

35. The mating structure of claim 32, further including alignment mechanism to spatially align between the coupler matrix and the receiving matrix, at least one fixation pin is configured for electrical coupling with at least one respective hole.

36. The mating structure of claim 32, wherein the alignment mechanism is configured to align between said coupler matrix and said receiving matrix by pressing one against another.

37. The mating structure of claim 32, wherein said coupling matrix is structurally associated with at least one conical bulge configured to enter at least one respective conical cavity structurally associated with the receiving matrix to facilitate three-dimensional alignment of coupling matrix with the receiving matrix.

38. A biopsy system comprising:
(a) a handle for receiving a removable biopsy device;
(b) said removable biopsy device comprising:
an inner needle comprising a tip configured to pierce tissue and a tissue sensing unit for characterizing tissue properties, at least part of said tissue sensing unit being positioned on the inner needle,
a recess formed longitudinally in said inner needle proximal to the tip and configured for receiving a tissue portion from a region of interest;
an outer sheath configured to change position relative to the inner needle between a forward position of the sheath in which the sheath totally covers the recess and a backward position of the sheath in which the recess and the at least part of the tissue sensing unit are uncovered, the outer sheath comprising a cutting edge at its distal end configured to cut and enclose the tissue portion into the recess when moving relatively to the needle from the backward position to the forward position; and
a connector element configured for receiving sensing signals from said tissue sensing unit and delivering the signals to a control unit using the mating structure of claim 32.
